(19) United States
(12) Patent Application Publication
(10) Pub. No.: US 2024/0366707 A1
(43) Pub. Date: Nov. 7, 2024

(54) COMPOSITION FOR THE PREVENTION AND/OR TREATMENT OF GASTRIC AND ESOPHAGEAL DISEASES

(71) Applicant: NEILOS S.R.L., Piano di Sorrento (IT)

(72) Inventor: Umberto DI MAIO, Piano di Sorrento (IT)

(73) Assignee: NEILOS S.R.L., Piano di Sorrento, NA (IT)

(21) Appl. No.: 18/687,134

(22) PCT Filed: Aug. 25, 2022
(86) PCT No.: PCT/IB2022/057961

§ 371 Date: Feb. 27, 2024

(30) Foreign Application Priority Data
Aug. 27, 2021 (IT) 102021000021995

(51) Int. Cl. A61K 36/736; A61K 31/737; A61K 35/644; A61P 1/04

(52) U.S. Cl. CPC A61K 36/736; A61K 31/737; A61K 35/644; A61P 1/04

(57) ABSTRACT

The present invention relates to a composition for oral use comprising chondroitin sulphate, an extract of Prunus dulcis and optionally honey, for the treatment of gastric and esophageal diseases, in particular of the gastroesophageal reflux disease.

### FIELD OF THE INVENTION

The present invention relates to a composition for oral use comprising chondroitin sulphate, an extract of Prunus dulcis and optionally honey for the treatment of gastric and esophageal diseases, in particular of the gastroesophageal reflux disease.

Such invention is based upon the synergic action of the above-mentioned active principles.

### STATE OF ART

Under gastroesophageal reflux “the unvoluntary and unconscious passage of a portion of the gastric content in the esophagus, without involving the gastric and abdominal muscles” is meant. The esophagus is a 25-30 long channel connecting the mouth with the stomach, along its path it is possible to detect two sphincter structures: the first one between hypopharynx and esophageal cervical tract (Upper Esophageal Sphincter, UES), the second one, Lower Esophageal Sphincter (LES), at the level of the esophagus-gastric junction. The latter is a high-pressure area representing the main anti-reflux structure, thanks to its localization between the intrathoracic negative pressure and the intra-abdominal positive pressure area. Then, under normal conditions, an increase in the abdominal pressure has an impact at the LES level by preventing the ingested material from returning into esophagus. Under physiological conditions LES is closed and it is released for a period of time of about 3-10 seconds after swallowing.

Other anatomical structures, apart from LES, contributing to keep the anti-reflux barrier are:
His angle, acute angle formed between esophagus and gastric fundus;
the phrenico-esophageal ligament;
the diaphragmatic collar, consisting of diaphragm beams, which arranging like a scarf around the esophagus throttle the lumen thereof during the inspiratory phase.

To the pathogenesis of the gastroesophageal reflux disease (GERD) several factors belong, such as for example:
1. Insufficient anti-reflux barrier of the lower esophageal sphincter which has the purpose of preventing mechanically the gastric juices from flowing again into the esophagus.
2. Delay in the gastric emptying, due to anatomical anomalies or functional alterations: (i) anatomical anomalies: stenosis of pylorus (it is the stomach's terminal region, adjusting the passage of the gastric content into the duodenum): (ii) functional alterations: motor alterations of fundus (region responsible for emptying the liquids)
3. Insufficient mechanism of esophageal clearing, which has the purpose of minimizing the contact between esophageal mucosa and gastric juices acting both through esophageal peristalsis and through neutralization of the acid residues thanks to saliva.
4. Gastric hyperacidity.
5. Agressivity of the gastric content which flows again into the esophagus, due to the action of the hydrochloric acid.
6. Duodenal-gastric reflux with passage in the stomach of pancreatic-biliary secretions which, in case of gastroesophageal reflux, can determine more severe lesions.

Among other predisposing factors there is smoke, incorrect dietary-behavioural habits (abundant meals, food rich in fats, caffeine), drugs, pregnancy and obesity can exacerbate GERD. Even hiatal hernia (passage of a stomach portion inside the thorax, through a hole in the diaphragm called esophageal hiatus) is often associated to GERD and it can contribute to the prolonged exposure to the gastroduodenal content. Generally, the esophageal hiatus' walls are well adherent to esophagus, but it may happen that the anchoring structures of the lower portion of esophagus lose tone, by favouring the going back of a small stomach portion in the thorax.

Whatever the cause may be, the frequent and repeated contact of the gastric material regurgitated with the gastric mucosa exerts on the latter a harmful action which is the more serious the longer the contact time is and the lower the reflux pH is. The persistent phlogistic action due to the esophageal mucosa becomes over time responsible for the inflammatory reaction which can evolve in ulcerations, in stenosis and in the so-called columnar metaplasia (or Barret epithelium, most important single risk factor for developing esophageal adenocarcinoma). The considered typical symptoms are represented by the retrosternal heartburn (defined by the patient as burning sensation beginning at the stomach or at the lower portion of the thorax and which goes back towards the neck) and by regurgitation (perception of liquid with bitter and acid taste inside the oral cavity), symptoms the specificity thereof for GERD is equal to 89 and 95%, respectively. Frequent, but less specific, symptoms are odynophagia, dysphagia, eructations, epigastric pain, swelling, digestive difficulty. Some of these symptoms characterize functional dyspepsia diagnosis and it is known that ±1 between 10% and 17% of patients requesting medical intervention due to dyspepsia have GERD.

GERD is one of the pathological conditions most frequently encountered by gastroenterologists.

From a study on the prevalence of the disease it was found that GERD has a prevalence of 10-20% in the Western countries against only 5% found in Asia; in particular a greater number of cases was found in North America then in North Europe and South Europe.

Scientific studies demonstrate that the disease symptoms have a strong impact on the quality of life since the persistent reflux symptoms, even during the treatment with inhibitors of the protonic pump for example, are associated to a reduced physical and mental well-being.

Since it is a chronic disease, the conventional treatment is almost always long lasting and, depending upon gravity, it consists in changes in the life style (eliminating chocolate, caffeine, alcohol, cigarette smoke, weight loss, etcetera), pharmacological treatments, surgical therapy.

The classes of drugs commonly used in GERD include: antiacid drugs, antagonist of H2 receptor of histamine and protonic pump inhibitors (PPI), prokinetic agents.

The antiacids are over-the-counter drugs offering a quick relief of symptoms of the disease, but they are not able to induce a curative effect in the erosive esophagitis. These drugs include carbonates or bicarbonates which reduce the stomach acidity by reacting with the hydrochloric acid by releasing carbon dioxide.

The antagonist H2 drugs such as ranitidine, famotidine, cimetidine, guarantee a temporary relief of the symptoms although with a slower time of onset than the antiacids. The use for prolonged time periods is not recommended since the patients could develop tolerance within 1-2 weeks and however the effect of these drugs is not of curative type. The PPI drugs (pantoprazole, lansoprazole, omeprazole, etcetera) represent the standard treatment in the gastroesophageal reflux diseases, in fact the number of prescriptions of such drugs has doubled over the last 10 years. Often such prescriptions are associated to those of anti-inflammatory drugs of steroid or not steroid nature. The action mechanism of PPIs includes blocking the protonic pump at the level of the parietal cells of the stomach; this APTasic hydrogen/potassium pump determines the release of hydrochloric acid in the stomach lumen. With respect to the antagonist H2 drugs, these drugs have a quicker effect and above all they exert a curative effect on the esophagus lesions. The side effects mostly found in the treatment with PPI are nausea, diarrhea, headache, insomnia and anaphylactic reactions.

The prokinetic agents, such as cisapride or metoclopramide, activate the receptors of serotonin or dopamine capable of increasing the esophageal or gastric peristalsis. These drugs have a slow action onset, a short duration and they have no curative effect on the disease. Moreover, they have different side effects such as tremors, dyskinesia, fatigue and increase in adverse events at heart level therefore their use is quite limited in the GERD treatment.

Apart from the traditional pharmacological remedies, even alginates are used for the symptomatic treatment of GERD. Alginates, such as alginate sodium, are natural polysaccharides which, when in contact with the gastric environment, precipitate by forming, in few minutes, a low-density gel. The ph variation triggered by the bicarbonates and carbonates, almost always present too in the formulations on the market, release carbon dioxide which is trapped inside the alginate gel, by making it to float on the gastric content. The alginate gel forms in the stomach portion near the gastroesophageal junction, exactly where the acid pocket develops. In this way the going back of acid from the stomach to the esophageal channel is blocked or strongly reduced.

The purpose of the present invention is to provide a composition alternative to those known in the state of known art useful in the treatment for treating gastric and esophageal diseases, in particular the gastroesophageal reflux disease.

### SUMMARY OF THE INVENTION

The present invention is based upon the search for and identification of a new combination of active principles exerting the synergic action, strengthened with the various components of the combination the invention relates to.

The present invention relates to compositions comprising or consisting of a mixture of chondroitin sulphate and an extract of Prunus dulcis and optionally honey. The present invention further relates to such compositions for use in the treatment of gastric and esophageal diseases, in particular of gastroesophageal reflux disease.

The present invention provides in a single composition the following advantages:
chondroitin sulphate creates a mechanical barrier protecting mucosa and favouring the regeneration process.
The extract of Prunus dulcis is capable of adhering to the mucosae with hydrating and soothing effect by favouring at the same time the physiological digestion process;
honey, if present, guarantees an anti-inflammatory action.

Other advantages and features of the present invention will result evident from the following detailed description.

### DETAILED DESCRIPTION OF THE INVENTION

The present invention describes a composition comprising as main active ingredients chondroitin sulphate and an extract of Prunus dulcis; the composition can optionally include even honey.

Chondroitin sulphate is a substance of natural origin present in almost all organisms, both vertebrates and invertebrates. In the human body it is a fundamental component of the extracellular matrix and has a very important role in the health and cellular wellbeing of tissues such as cartilages, tendons, ligaments, skin and blood vessels.

From the chemical point of view, it belongs to the class of glycosaminoglycans.

There is increasing evidence that chondroitin sulphate has important biological functions. These effects are correlated to the capability of chondroitin sulphate to interact with a huge variety of molecules such as growth factors, protease inhibitors, cytokines, chemokines, adhesion molecules and at last with pathogen virulence factors. In fact, it is involved in the processes of proliferation, differentiation and cellular migration, tissue morphogenesis, organogenesis, reparation of wounds and, at last, inflammation regulation.

Among the various activities attributed thereto there is the capability of binding with pepsin and inhibiting its activity and in fact, in past chondroitin was evaluated for the treatment of peptic ulcer.

In the last few years various studies were performed to demonstrate the effectiveness of chondroitin sulphate in smoothing or removing the symptoms associated to the gastroesophageal reflux, through the formation of a mechanical barrier avoiding the contact between mucosa and gastric juice, highly acid and in large part root cause of the disease.

In an ex vivo study by utilizing the esophageal lumen of pig in which a damage to the mucosa was induced to evaluate the difference in permeability before and after the treatment with an association containing chondroitin sulphate and hyaluronic acid. In this study it came to the conclusion that the use of the association chondroitin sulphate and hyaluronic acid is capable of reducing significantly the mucosa permeability by acting by means of the formation of a mechanical barrier.

In another study one evaluated the capability of chondroitin sulphate, if administered in association with hyaluronic acid and a muco-adhesive substance, to improve the condition in patients with gastroesophageal reflux of not erosive type by evaluating TSS (Total Symptom Score) which takes into consideration the symptoms such as stomach ache, sour taste, retrosternal pain and by evaluating the quality of life by filling-in the SF 36 test. The study, which was performed in randomized placebo-controlled double blind on a sample of 154 patients, provided the administration of chondroitin sulphate in association with hyaluronic acid or the administration of a placebo twice a day for 14 days in addition to the usual therapy with pump or anti H2 inhibitors. At the end of the treatment, a significant decrease in the total symptoms was obtained in 50% of the patients treated with the association with respect to a decrease in the symptoms in only 30% of the patients thereto the placebo was administered. Moreover, a significant improvement in the score on the questionnaire of the life quality, even if not significantly different from placebo, was obtained.

In a placebo-controlled study the potential protective role of chondroitin sulphate in the gastroesophageal reflux disease was evaluated, if administered in association with hyaluronic acid. This study was performed in placebo-controlled cross-over double blind on a sample of 20 patients divided in the treatment group and in the placebo group. The treatment provided the administration of a spoon of the syrup containing chondroitin sulphate and hyaluronic acid every 8 hours away from meals during the day, and two spoons before bedtime. Various parameters were taken into consideration thereamong SSSI (Sum of Symptoms Score intensity), SSID (Symptoms Score Intensity Difference), difference in intensity of the stomach ache and acid regurgitation, the complete disappearance of the symptoms and the degree of patients showing a quick action. At the end of the study a significant decrease in the parameters SSSI, SSID, ache intensity and in acid regurgitation was obtained. Moreover, the treatment caused a high degree of disappearance of symptoms and even the time used to obtain the effect was reduced, by demonstrating the effectiveness of the association chondroitin sulphate and hyaluronic acid in inducing a quick relief of the classical symptoms associated to reflux.

From these studies it is clear that chondroitin sulphate has the capability of protecting the mucosa, preventing the damage induced by an excessive gastric acidity and consequently that it can be used in therapy as co-adjuvant treatment in the gastroesophageal reflux disease.

The almond tree (Prunus dulcis) is an important fruit tree originating in central Asia, currently produced all over the world. The fruit seed is known as almond constituting the edible portion, it is a seed formed by two big cotyledons coated with a brown peel and protected by an external shell with an intermediate shell.

The main substances found in the fruit, apart from water, are lipidic fraction (40-67%), followed by protein fraction, carbohydrates, minerals and vitamins. The oil extracted from almonds mainly consists of Monounsaturated fatty acids (MUFA) which associate to the reduction in LDL cholesterol. The protein fraction consists of Globulins and Albumins; the main free amino acids are the glutamic acid and the aspartic acid, followed by arginine. The total content of carbohydrates varies from 14% to 28%. The almonds are characterized by a low content of soluble sugars, in a range from 2.6% to 7.9%, most sugars are not reducing and the sucrose represents more than 90% of the total sugars. As far as the content in minerals are concerned, specifically for the macro minerals, potassium is the main element followed by phosphorus, whereas among the micro elements, those mostly present are sodium, chlorine, iron, copper, zinc and manganese. Vitamin E, in particular α-tocopherol showing a strong antioxidant activity, is the most abundant one in the almond oil; moreover, the almond fruit is a good source of Vitamin B1, B2, B6 and Niacin. Moreover, several polyphenols (about 312 mg/100 g of fruit) were identified; the most abundant ones are hydrolysable tannins, pro-anthocyanidins and flavonoids.

Several studies showed anti-oxidant and scavenger activity of the free radicals for the almond extracts; the presence of huge amounts of phenolic compounds can be attributed to the antioxidant activities. The intestinal mucosa is vulnerable to oxidative stress due to constant exposure to the reactive oxygen species (ROS) generated by the lumen content. The oxidative stress can cause cellular damages directly or through alteration of signalling routes. In an in vivo study, inflammatory intestinal colitis was induced in mice which subsequently were treated with extracts of almonds. The treatment reduced the appearance of diarrhea and weight loss; this was associated to a significant reduction in the activity of myeloperoxidase in colon. Moreover, it reduced the release of pro-inflammatory cytokines, the appearance of i-NOS, nitrotyrosine and PARP in colon and it reduced the up-regulation of ICAM-1 and the expression of P-selectin (Mandalari, 2011).

The polyphenols extracted from almond have a potential antimicrobic activity, in particular it was demonstrated that they are active towards Listeria monocytogenes and Staphylococcus aureus, whereas Salmonella enterica and Helicobacter pylori demonstrated to be sensitive to the extracts of the almond peels. The prebiotic effects of the almond extracts were studied in vitro by using mixed faecal bacterial cultures and it was found that, after digestion of almonds, the populations of Bifidobacteria and rectal Eubacterium increased significantly. These results were confirmed even in in-vivo and clinical studies, where it was demonstrated that after 6 weeks of treatment the Bifidobacterium spp. and Lactobacillus spp. populations in the faecal samples increased significantly, whereas Escherichia coli remained stable and Clostridium perfringens was significantly removed. These changes in intestinal bacterial population induce variations in the activity of enzymes, in particular the activity of β-galactosidase increases, and the activity of β-glucuronidase, nitroreductase and azoreductase reduces; the reduction in the activity of glycosidase can induce a failure in the metabolism of the not absorbed carbohydrates, like the one shown in the ulcer colitis and in Crohn disease. The intestinal microbiota improvement induces beneficial effects for the human health, such as the activation of the immune response, the production of bacteriocins, nutritional and physical competition with pathogens and the maintenance of an acid environment.

The anti-inflammatory potential of almonds was studied through a randomized study on subjects who consumed almonds with respect to a placebo group. After the treatment period, the levels of E-selectin, adhesion molecule involved in the inflammation process, resulted to be significantly lower in the group who used almonds with respect to placebo. According to the authors, the anti-inflammatory effects could be attributed mainly to the high content of monounsaturated fatty acids (MUFA), which was considered responsible for the decrease in the levels of E-selectin and reactive C protein.

Honey is a substance containing about 200 different components, mainly sugars and water, and other substances like proteins, organic acids, vitamins and minerals, phenolic compounds and volatile compounds.

The monosaccharides, glucose and fructose represent about 75% of the sugars found in honey, followed by 10-15% of disaccharides; sugars are responsible for properties like energy value, viscosity, hygroscopicity and granulometry.

The protein content varies based upon the production species, for example honey from Apis mellifera has a protein content going from 0.2 to 1.6%, but it varies even based upon the different storage conditions. In honey, proline represents 50-85% of amino acids, it was used as criterion for evaluating honey ripening and, in some cases, of adulteration with sugar. All honeys have a light acidity, due to the presence of about 0.57% of organic acids.

Honey includes small amounts of vitamins, especially the vitamin complex B; the content of minerals in honey varies from 0.04%, in light honeys, to 0.2% in dark honeys. Potassium is the most abundant element, generally corresponding to one third of the total mineral content which can be found in honey, whereas in reduced amounts it contains sodium, iron, copper, manganese, calcium and magnesium.

The main functional components of honey are flavonoids. They can significantly contribute to the total antioxidant activity of honey, bringing beneficial effects for the human health.

The antioxidant activity of the flavonoids in most cases depends upon the number and position of the hydroxyl groups and other substituents, and upon the glycosylation of flavonoids. In honey there are complex mixtures of volatile compounds, responsible for the taste of honey, of different chemical families, belonging to monoterpenes, sesquiterpenes, benzene derivatives, and reduced content of alcohols, esters, fatty acids, ketones and aldehydes.

Honey is historically used to treat various affections in the traditional medicine of various cultures. Classically, it is used to favour healing of wounds, due to various causes such as burns, diabetic ulcers or still scars after surgical operations and to avoid possible development of bacterial infections in the latter.

For this reason, over the last years, honey, its composition, and its biological effects have been extensively studied and tested.

From the performed researches a huge amount of scientific evidence was obtained, supporting the use of honey in this application field, above all by demonstrating the action mechanisms therethrough it succeeds in performing a positive biological effect on healing wounds if applied topically.

In particular, it was clarified that honey does not act simply through a mechanical-physical effect linked to the properties of the latter, which make it, among other things, an ideal compound as material for covering wounds, but it acts also through various biological activities due to the presence of bioactive substances, which can intervene and improve the healing process.

When it is applied topically over a wound, honey impacts positively on the wound environment and on the healing process thanks to its physical properties, since honey generally has an acid pH around 3.2-4.5 and it is well known that an acid environment in the wounds favours the release of oxygen from hemoglobin, thus increasing the amount of oxygen available inside the wound. Additionally, the acid environment disadvantages the activity of the proteolytical enzymes, by consequently decreasing the degradation of the extracellular matrix, fundamental for the tissue repair process.

As said previously, honey has a very high content of sugars, this confers the substance a high osmolarity, which results to be positive in healing wounds. This because, through the osmotic effect induced by the application of honey, water is drawn from the wound and, consequently, the accumulation of liquids and the formation of oedema are avoided. Moreover, the sugars even draw water from the bacterial cells present inside the wound, thus by avoiding the growth and the formation of bacterial colonies. This, at least until there is an excessive dilution of honey.

Additionally, it is important to consider that due to the high viscosity a protective barrier above the injured skin, almost impenetrable by external agents, is created.

However, as said previously, the important activity performed by honey in the damage prevention and in healing can be attributed only partially to the physical properties of the substance. In fact, more and more studies attribute anti-bacterial, anti-inflammatory and immunomodulating activities to the various components found inside honey.

The anti-inflammatory activity has an additional important role in honey action mechanism. This because there is a reduction in oedema and exudate, pain calming and reducing effect.

In particular, various clinical studies evaluated honey effectiveness in improving burns, by comparing its activity with that of silver sulfadiazine, which acted as positive control.

Honey application decreased the levels of the inflammatory markers, decreased the levels of malondialdehyde, and reduced the number of inflammatory cells present in biopsies of wounds.

Additionally, in order to demonstrate that the effect is directly caused by an anti-inflammatory action and not by a secondary effect due to the anti-bacterial activity, various studies were performed on animal models in which the wound was produced and maintained under aseptic conditions, and even under these conditions honey demonstrated its anti-inflammatory activity.

Moreover, honey demonstrated to be effective as anti-inflammatory agent, if applied on the mucosa in which a damage was present, induced by radiotherapy, and just as effectively it decreased the inflammatory conditions when applied on the rat ophthalmic mucosa, in which a corneal abrasion or a keratitis induced by endotoxin was present.

This by now well established and recognized anti-inflammatory activity generally is attributed to the existing phenolic compounds, compounds which have the capability of inhibiting the production of pro-inflammatory cytokines such as for example TNF-α.

Thanks to the anti-inflammatory and anti-bacterial biological activities which were attributed to it and which were demonstrated, and thanks to the physical-chemical properties, allowing it to create a protective mechanical barrier on the mucosa, it is plausible thinking that honey could be an important weapon to fight the symptoms of the gastroesophageal reflux, since it could both create the mechanical barrier effect, and then avoid the contact between mucosa and the highly acid gastric content, and explicate an anti-inflammatory and soothing effect, and then bring an additional advantage and decrease the symptoms associated to the disease.

The inventors have observed that most synergic action among the different active ingredients is present with the following concentrations, meant as amounts per dosage unit:
chondroitin sulphate is present in a concentration by weight comprised from 0.5 mg to 8000 mg, preferably from 1 mg to 6000 mg;
the extract of Prunus dulcis is in a quantity by weight comprised from 0.5 mg to 5000 mg, preferably from 1 mg to 3500 mg;
honey, if present, is present in a quantity by weight comprised between 1 mg and 4000 mg, preferably from 5 mg to 2500 mg.

The compositions according to the present invention can be formulated under any form and route of administration and associated to any other component, in a variety of ways, preferably they will be formulated for oral use for example as capsules, soft capsules, tablets, pills, gelatines, powders or granules, solutions, suspensions, gels, syrups, elixirs. Such excipients can be selected for example among those usually known in the state of art and include, without being limiting thereto: a) carriers, such as for example sodium citrate and calcium phosphate, b) fillers such as for example starch, lactose, microcrystalline cellulose, sucrose, glucose, mannitol and colloidal silica, c) humectants, such as for example glycerol, d) disintegrating agents, such as alginates, calcium carbonate, starches, derivatives of starch, of cellulose and of polyvinylpyrrolidone, silicates and sodium carbonate, e) binders such as carboxymethylcellulose, alginates, gelatine, polyvinylpyrrolidone, sucrose, polymeric derivatives of cellulose, starch derivatives, f) retarding agents such as paraffin, cellulose polymers, esters of fatty acids, g) absorption accelerators, such as compounds of quaternary ammonium, h) wetting agents and surfactants such as cetyl alcohol and glycerol monostearate, i) adsorbents, such as Benthic clays and kaolin, k) lubricants such as talcum, calcium stearate, magnesium stearate, polyethylene glycol, sodium lauryl sulfate, sodium stearyl fumarate, j) glidants such as talcum, colloidal silica.

The forms of solid dosage, such as tablets, capsules, soft capsules, gelatines, pills and granules, could be coated with enteric, gastric coatings or coatings of other type known in the state of art. They can include matting agents and they can be of the type to allow the release of the active ingredients only or preferably in a certain tract of the intestine, in case, in delayed way. Substances which can allow such delayed use include, but they are not limited thereto, polymers and waxes.

The soft capsules could house the antioxidant active substances in liquid form alone or in solutions, suspensions or emulsions of the active substances in a liquid solvent. The soft capsules could be characterized by a casing qualitatively similar to that of the hard ones, but thicker and softer.

Liquid forms suitable to an oral administration for example are emulsions, solutions, prepared or extemporary suspensions, syrups and elixirs. Excipients suitable to the formulations according to the present invention in liquid forms for oral use include, but they are not limited thereto, diluents such as water or other solvents, solubilizing agents and emulsifiers selected among ethyl alcohol, polyalcohols, propylene glycol, glycerol, polyethylene glycol and sorbitan esters. These formulations can even include sweeteners and flavourings.

The compositions will be for example a medical device, food supplement, a nutraceutical, dietary and nutritional composition, food product, a beverage, a nutraceutical product, a medicament, medicated food, food for special medical purposes, food. The compositions will be mainly intended to be used by human beings, but they could also be used on animals.

The combination of the above-mentioned active ingredients could be used formulated in one single composition according to the various above-described embodiments or in a kit containing the different separated ingredients, for example in single compositions as capsules, pills, tablets for sequential or contemporary administration of the different ingredients.

The above-described compositions and kits could be used/administered/taken for the treatment of gastric and esophageal diseases, in particular of the gastroesophageal reflux disease.

### EXAMPLES

Some not limiting examples of daily dosages of the combination of active ingredients used in the compositions of the present invention are shown hereinafter.

### Example 1

| Active principle | Dose per dosage unit |
|---|---|
| Chondroitin sulphate | 20 mg |
| Almond oil (Prunus dulcis) | 10 mg |

### Example 2

| Active principle | Dose per dosage unit | |
|---|---|---|
| Chondroitin sulphate | 200 | mg |
| Almond oil (Prunus dulcis) | 10 | mg |
| Honey | 5 | mg |

### Example 3

| Active principle | Dose per dosage unit | |
|---|---|---|
| Chondroitin sulphate | 100 | mg |
| Almond oil (Prunus dulcis) | 50 | mg |
| Honey | 30 | mg |

### Example 4

| Active principle | Dose per dosage unit |
|---|---|
| Chondroitin sulphate | 50 mg |
| Almond oil (Prunus dulcis) | 60 mg |

### EXPERIMENTAL DATA

The authors of the present invention have observed that the simultaneous administration of the active ingredients selected thereby provides a more effective treatment of the gastroesophageal reflux disease in the subjects requiring it, with respect to a therapy providing the administration of the single active principles.

The synergic action of the single components is evaluated by in vitro or in vivo methods.

In vitro tests are for example the study of the viscosity depending upon temperature or other applicable parameters apt to demonstrate any improvement effect attributed to the synergy of the three components.

The synergic activity of the components can be evaluated in vitro through muco-adhesion tests performed on cells (for example on epithelial cells of mucosa) or by means of other validated methods (for example tilted plane with mucin).

On specific cellular model (for example gastric cells) it is possible to evaluate the antioxidant, anti-inflammatory activity of the composition the present invention relates to.

In vivo methods used to evaluate the anti-reflux effect of the formulation with respect to the single components are: gastric emptying, and/or reflux esophagitis, and/or gastric secretion, and/or gastric ulcer. The evaluation of such parameters is performed after administration of the single components, and association thereof, to animals.

The gastric emptying is performed on mice or rats thereto a suspension of phenol red in carboxymethylcellulose is administered. After about 20 minutes the animals are sacrificed in atmosphere saturated with CO₂ and the stomach is collected, which is positioned in a test tube of physiological solution. To each test tube an amount of NaOH is added to develop the maximum colour intensity. Subsequently, the spectrophotometric analysis (560 nm) is performed and the percentage of gastric emptying is obtained from the following formula:

$$100 \times ( 1 - [ amount of phenol red present in the stomach after 20 minutes ) / \\ ( amount of phenol red present in the stomach at time 0 ] ) .$$

Reflux esophagitis and the gastric secretion are induced by leaving free access to water to animals and fasting for 24 hours; subsequently the animals are anesthetized, the abdomen open and pylorus is tied. After about 4 hours from the surgical procedure the mice are sacrificed in atmosphere saturated with CO₂ and the stomach and esophagus are collected with the purpose of evaluating: the esophageal and gastric macroscopic damage, the esophageal and gastric inflammation level (myeloperoxidase activity), volume of the gastric content, pH and total acidity. Formalin is used as reference drug administered at a dose of 40 mg/Kg.

1. A composition for oral use comprising chondroitin sulphate and an extract of Prunus dulcis.

2. The composition according to claim 1, further comprising honey.

3. The composition according to claim 1, in a form selected from capsule, soft capsule, tablet, pill, gelatine, powder, granule, emulsion, solution, suspension, gel, or syrup.

4. The composition according to claim 1, wherein:
chondroitin sulphate is present in a quantity by weight from 0.5 mg to 8000 mg per dosage unit; and
the extract of Prunus dulcis is present in a quantity by weight from 0.5 mg to 5000 mg per dosage unit.

5. The composition according to claim 2, wherein honey is present in a quantity by weight between 1 mg and 4000 mg per dosage unit.

6. The composition according to claim 1, wherein said composition is a medical device, a food supplement, a nutraceutical, dietary and nutritional composition, a food product, a beverage, a nutraceutical product, a medicament, medicated food or food for special medical purposes.

7. A method of treating gastric and esophageal disease in a subject, comprising administering a therapeutically effective amount of the composition according to claim 1 to the subject in need thereof.

8. The method according to claim 7, wherein said disease is gastroesophageal reflux.

9. A kit comprising chondroitin sulphate and an extract of Prunus dulcis.

10. The composition according to claim 4, wherein chondroitin sulphate is present in a quantity by weight from 1 mg to 6000 mg per dosage unit and the extract of Prunus dulcis is present in a quantity by weight from 1 mg to 3500 mg per dosage unit.

11. The composition according to claim 5, wherein honey is present in a quantity by weight from 5 mg to 2500 mg per dosage unit.

12. The kit according to claim 9, further comprising honey.
